(19) United States
(12) Patent Application Publication
(10) Pub. No.: US 2023/0381508 A1
(43) Pub. Date: Nov. 30, 2023

(54) Layered Stimulus Patterning to Synergistically Optimize Brain Clearance at Multiple Points in Clearance System and Real-Time Dial to Change Drug Delivery Profiles

(71) Applicant: Wisconsin Alumni Research Foundation, Madison, WI (US)

(72) Inventors: Kip Ludwig, Middleton, WI (US); Justin Williams, Cambridge, WI (US); Kevin Cheng, Verona, WI (US); Nishant Verma, Madison, WI (US)

(21) Appl. No.: 18/201,881

(22) Filed: May 25, 2023

Related U.S. Application Data
(60) Provisional application No. 63/346,038, filed on May 26, 2022.

(51) Int. Cl. A61N 1/36; A61N 1/04

(52) U.S. Cl. CPC A61N 1/36025; A61N 1/0456; A61N 1/36034

(57) ABSTRACT

Administration of low frequency electrical stimulation of the cranial nerves delivered during sleep increases the presence and function of aquaporin-4 (AQP4) channels in the astrocytic endfeet surrounding descending arterioles in the brain. This underlying low frequency stimulation pattern is overlaid with temporally patterned ‘bursts’ of higher frequency stimulation to pulse the underlying artery to drive cerebrospinal fluid (CSF) penetration into the parenchyma. This also serves to create more movement in general within the parenchymal extracellular space to increase the probability of waste biomolecules to interact with sites for active transport out of the brain. During the period of sleep, these two layered patterns will be periodically replaced with multiple continuous periods of stimulation at gamma frequency to promote a more phagocytic phenotype in glial cells to help break down waste biomolecules and misfolded proteins for subsequent clearance. Administration of electrical stimulation can be selectively modified to adjust CSF clearance, for example, to quickly clear drug concentrations in the brain during an overdose.

### CROSS-REFERENCE TO RELATED APPLICATIONS

This application claims the benefit of U.S. Provisional Patent Application No. 63/346,038, filed May 26, 2022, which is incorporated by reference herein in its entirety.

### STATEMENT REGARDING FEDERALLY SPONSORED RESEARCH OR DEVELOPMENT

This invention was made with government support under N66001-17-2-4010 awarded by the DOD/DARPA and under EB029251 awarded by the National Institutes of Health. The government has certain rights in the invention.

### BACKGROUND OF THE INVENTION

The present invention relates to electrical stimulation of target nerves to enhance waste clearance in the brain.

Waste removal from the central nervous system (CNS) is essential for maintaining brain homeostasis. Disruption of waste clearance can lead to protein accumulation. The aggregation of pathogenic proteins β-amyloid, α-synuclein, and C-tau in the brain may cause the deleterious effects of numerous diseases and disorders such as traumatic brain injury/chronic traumatic encephalopathy, epilepsy, Alzheimer's disease, and Parkinson's disease. Removal of these pathogenic proteins has been found to have substantial therapeutic benefit, for example, in treating traumatic brain injury/chronic traumatic encephalopathy, epilepsy, Alzheimer's disease, and Parkinson's disease. Disruption of waste clearance is also implicated in several mental health disorders including depression, bipolar disorder (BPD), and anxiety.

The glymphatic system, or glymphatic clearance pathway, is a macroscopic waste clearance system for the vertebrate CNS utilizing a unique system of perivascular tunnels formed by glial cells to promote efficient elimination of soluble and insoluble proteins and metabolites from the CNS.

Specifically, recent studies have shown that cerebrospinal fluid (CSF) and interstitial fluid (ISF) continuously interchange to clear pathogenic proteins and metabolic byproducts from the brain. This exchange is facilitated by convective influx of CSF along paravascular spaces, also known as Virchow-Robin spaces, which are defined by the outer-wall of cerebral vessels and a glial sheath formed primarily by astrocytic endfeet. From the subarachnoid space CSF is driven down along the Virchow-Robin space and into the brain parenchyma though a combination of arterial wall movement, respiration, and CSF pressure gradients. The subsequent transport of CSF into the brain parenchyma is mediated by aquaporin-4 (AQP4) water channels whose distribution is highly polarized towards the astrocytic endfeet. The movement of CSF through the brain parenchyma further drives convective ISF fluxes within the tissue toward the perivenous spaces eventually draining from the brain via meningeal lymphatic vessels, along cranial nerve sheaths, and through the deep cervical lymph nodes. Therefore, the glymphatic system has been found to play an important role in clearing pathogenic proteins and metabolic byproducts from the brain, separate from the BBB.

In addition to facilitating clearance of metabolic waste and disease associated molecules from the brain, increasing the penetration of CSF into the brain parenchyma can serve many therapeutic purposes, including diluting endogenous neurochemical transmitter concentrations within the brain, altering the clearance rates of drugs delivered orally that penetrate through the blood brain barrier or delivered via a catheter system to the brain, and reducing non-synaptic (ephaptic) coupling between neurons to treat diverse conditions including anxiety disorders, tremor, and seizure.

### SUMMARY OF THE INVENTION

The present inventors have found that arterial wall movement generated by smooth muscle cells creates pulse waves driving the entry of CSF along the paravascular space. This wall movement can be increased by modulating arterial sympathetic and parasympathetic tone, respiration, and cardiac pulse rate. Wall movement refers to vessel wall dynamics such as pulsatility, vasodilation, and vasoconstriction which occur in the paravascular space and may contribute to pushing CSF along the paravascular space and into the parenchyma.

The present inventors have also found that convective fluid fluxes are elevated during periods of strong low frequency spectral power electroencephalogram (EEG) such as those that occur during the sleep state. This relationship is due to promotion of AQP4 mediated CSF/ISF exchange due to low frequency electric field effects on lipid physiology moderating AQP4 permeability. In addition, neuronal cell bodies increase in volume during periods of high activity and shrink during periods of low activity. This shrinkage during high spectral power low-frequency activity increases the extracellular fluid volume thereby decreasing the brain parenchyma's tortuosity and hydrostatic resistance to fluid movement making the microenvironment more conducive to CSF/ISF fluid flux. This fluid flux can be promoted by artificially driving low-frequency oscillations via electrical stimulation.

Therefore, the present inventors have found that 1) inducing arterial wall movement, 2) modulating AQP4 channel physiology, and 3) reducing neural activity to increase fluid in the extracellular space reducing resistance to fluid movement play a role in CSF/ISF exchange.

The present inventors have also found that electrical stimulation at gamma band frequencies promotes phagocytic phenotypes of glial cells. These cells digest cellular debris and help breakdown waste molecules to maintain a healthy microenvironment. Promoting the phagocytic activity of these cells prevents the accumulation and build-up of misfolded proteins associated with neurodegenerative disease.

Therefore, the present inventors propose a method of synergistically facilitating the exchange of CSF and ISF by creating a layered electrical stimulus pattern that induces cerebral arterial wall movement and promotes AQP4 mediated CSF/ISF exchange during sleep. Moreover, separating the layered stimulation pattern with a high frequency stimulus after sleep helps to break down waste biomolecules and misfolded proteins for further CSF clearance.

1) Synergistic Electrical Stimulation Protocols

Specifically, the present invention provides for the administration of low frequency electrical stimulation of the cranial nerves delivered during sleep to enhance CSF/ISF exchange across the astrocytic endfeet surrounding descending arterioles in the brain that serve as the “gate” to allow CSF into the parenchyma from the perivascular space. Promotion of CSF/ISF exchange at the perivenous space which drains the waste solute containing ISF from the parenchyma also enhances overall clearance activity. This underlying low frequency stimulation pattern is overlaid with temporally patterned “bursts” of higher frequency stimulation to pulse the underlying artery to drive CSF penetration into the parenchyma. This also creates more movement within the parenchymal extracellular space to increase the probability of waste biomolecules interacting with sites for active transport out of the brain. During or after the period of sleep, these two layered patterns will be periodically replaced with multiple continuous periods (e.g., about 15 minutes in duration) of stimulation at gamma frequency to promote a more phagocytic phenotype in glial cells to help break down waste biomolecules and misfolded proteins for subsequent clearance.

It is thus a feature of at least one embodiment of the present invention to 1) clear misfolded proteins whose aggregation is linked to the development of multiple age related neurodegenerative disorders, as well as secondary damage after traumatic brain injury and stroke, 2) improve cognitive function in healthy adults by clearing biomolecular waste from the metabolic processes of the brain, 3) restore normal brain homeostasis and waste clearance in individuals suffering from mental health disorders by rescuing glymphatic functions, and 4) increasing CSF penetration into the brain parenchyma to dilute endogenous neurochemical transmitters/hormones as well as sculpt the delivery profile of drugs to the brain.

One embodiment of the present invention provides an electrical stimulation device for stimulating cranial nerves to improve waste clearance through a glymphatic system or meningeal lymphatic system, the electrical stimulation device having an electrical generator generating a first carrier wave having a first carrier frequency stimulating the glymphatic or meningeal lymphatic system into increased cerebral spinal fluid (CSF)/interstitial fluid (ISF) flow and a second carrier wave having a first carrier frequency stimulating the glymphatic or meningeal lymphatic system into increased cerebral spinal fluid (CSF)/interstitial fluid (ISF) flow. A modulator receives the second carrier wave and a modulation wave to modulate the second carrier wave to produce a modulated second carrier wave. An electrical modulation generator generates the modulation wave having a predetermined periodicity providing a first period of stimulation eliciting vasocontriction and a second period of relaxation eliciting vasodilation to generate a pulsatile motion, the predetermined periodicity selected to increase arterial wall movement on top of enhanced glymphatic CSF/ISF fluid flux provided by continuous stimulation of the first carrier wave. At least one nerve stimulator is configured to stimulate a cranial nerve and receive the first carrier wave and the modulated second carrier wave simultaneously to stimulate the glymphatic system into increased cerebral spinal fluid (CSF)/interstitial fluid (ISF) flow.

It is thus a feature of at least one embodiment of the present invention to provide synergistic effect of both (1) increasing the wall movement of the arteries and (2) promoting AQP4 mediated fluid flux by delivering low frequency stimulation with higher frequency bursts to synergistically increase convective influxes of CSF along paravascular spaces in the brain.

The first carrier wave may have a frequency modulating the permeability of aquaporin-4 water channels.

It is thus a feature of at least one embodiment of the present invention to deliver low frequency stimulation (consistent with slow-wave sleep) that is associated with the control of AQP4 channel physiology.

The frequency of the second carrier wave may be at a frequency that is greater than the frequency of the first carrier wave.

It is thus a feature of at least one embodiment of the present invention to deliver bursts of higher frequency stimulation to pulse cerebral arterial vessels and drive CSF penetration.

A duty cycle of the modulation wave may be between 10% and 50%. The modulation wave may have an “ON” time duration that is at least twice as long as the total ON/OFF interval. The modulated second carrier wave has a duty cycle promoting arterial wall movement in the brain.

It is thus a feature of at least one embodiment of the present invention to introduce periods of relaxation into the electrical stimulation parameters to permit recovery processes of the dilated blood vessels.

The electrical generator may generate a third carrier wave and at least one electrode may receive the third carrier wave before or between periods of receiving the first carrier wave and modulated second carrier wave. The third carrier wave may have a third carrier frequency promoting phagocytic phenotype in glial cells to break down waste biomolecules and misfolded proteins.

It is thus a feature of at least one embodiment of the present invention to promote phagocytic phenotype in glial cells to break down waste biomolecules and misfolded proteins between the administration of temporally patterned bursts to clear the way for additional clearance. The third carrier wave may be at a frequency that is higher than a frequency of the first carrier wave.

It is thus a feature of at least one embodiment of the present invention to use high frequency during a patient's wakeful state to help improve break down of waste products.

The frequency of the third carrier wave may be at gamma frequency. The frequency of the third carrier wave may be centered around a frequency of approximately 25 to 200 Hz.

It is thus a feature of at least one embodiment of the present invention to increase the expression of pro-phagocytic genes and phenotypes in glial cells.

The third carrier wave may be delivered to the electrode prior to the delivery of the first and second carrier wave.

It is thus a feature of at least one embodiment of the present invention to activate microglia to phagocytosis (and accumulating misfolded proteins) and then subsequently clear out the metabolic waste products that phagocytosis produces.

At least one electrode may be adapted to stimulate at least one of a trigeminal nerve, buccal branch nerve, mental branch nerve, facial branch nerve, vagus branch nerve (e.g., auricular vagus nerve), cervical nerve, sympathetic trunk/sympathetic ganglia, and sympathetic efferent branches.

It is thus a feature of at least one embodiment of the present invention to create stimulus locked changes in cerebral blood flow as compared to stimulation of nerves where habituation occurs.

An alternative embodiment of the present invention provides a method of modifying waste clearance through a glymphatic system or meningeal lymphatic system including: positioning at least one electrode in close proximity to a nerve; generating a first carrier wave having a first carrier frequency stimulating the glymphatic or meningeal lymphatic system to increase cerebral spinal fluid (CSF)/interstitial fluid (ISF) flux; generating a second carrier wave having a second carrier frequency stimulating the glymphatic or meningeal lymphatic system into increased cerebral spinal fluid (CSF)/interstitial fluid (ISF) flow; generating a modulation wave having a predetermined periodicity providing a first period of stimulation and a second period of no stimulation, the predetermined periodicity selected to increase waste clearance over continuous stimulation of the glymphatic or meningeal lymphatic system by the first carrier wave; modulating the second carrier wave by applying the modulation wave to the second carrier wave; and applying the first and second carrier wave to at least one electrode simultaneously.

The method may further comprise generating a third carrier wave before or between periods of generating the first carrier wave and second carrier wave simultaneously and applying the third carrier wave to the electrode.

The first carrier wave and second carrier wave may be applied to a first electrode and the third carrier wave may be applied to a second electrode wherein the first electrode and second electrode are positioned at different locations to stimulate different nerves.

It is thus a feature of at least one embodiment of the present invention to provide optimal stimulation protocols by targeting different nerves that are most responsive to the desired physiological responses.

2) Real-Time Dial to Modulate Drug Delivery Profiles in the Brain

Additionally, the present invention provides a method of increasing or decreasing the exchange of CSF and ISF by altering the electrical stimulus protocol during certain drug delivery profiles. The electrical stimulation pattern may be activated to maximize the exchange of CSF and ISF when facile penetration of CSF into the parenchyma is desired to enhance drug delivery or drug clearance. In contrast, the electrical stimulation may be deactivated to minimize the exchange of CSF and ISF when slower penetration of CSF into the parenchyma is desired to allow for longer drug penetration and exposures (i.e., longer half-life of the drug) in the brain.

It is thus a feature of at least one embodiment of the present invention to provide a “real-time” dial to change the delivery profile of drugs that are orally administered that cross the BBB, or drugs that are delivered directly to the central nervous system via an implanted catheter.

One embodiment of the present invention provides a method of modifying waste clearance through a glymphatic system or meningeal lymphatic system including: positioning at least one electrode in close proximity to a nerve; generating a carrier wave having a carrier frequency stimulating the glymphatic or meningeal lymphatic system into increased cerebral spinal fluid (CSF)/interstitial fluid (ISF) flow; generating a modulation wave having a predetermined periodicity providing a first period of stimulation of the glymphatic or meningeal lymphatic system system and a second period of relaxation of the glymphatic or meningeal lymphatic system system, the predetermined periodicity selected to increase waste clearance over continuous stimulation of the glymphatic or meningeal lymphatic system by the carrier frequency; modulating the carrier wave by applying the modulation wave to at least one of the carrier wave; and selectively applying the carrier wave to at least one electrode wherein the carrier wave is delivered to the electrode when it is desired to increase waste clearance and the carrier wave is not delivered to the electrode when it is desired to decrease waste clearance.

It is thus a feature of at least one embodiment of the present invention to quickly alter CSF/ISF exchange in real-time (1) after drug administration to permit drugs to dwell in the brain longer or (2) to quickly remove drugs from the brain in cases of accidental or over delivery of drugs.

These particular objects and advantages may apply to only some embodiments falling within the claims and thus do not define the scope of the invention.

### BRIEF DESCRIPTION OF THE DRAWINGS

FIG. 1 is a schematic diagram of a human skull receiving electrical stimulation from electrodes positioned on facial and lingual nerves of the head in the tooth and jaw region and in the cheek region, in accordance with the present invention;

FIG. 2 is a schematic diagram of a human head receiving electrodes on or in a cheek region to deliver directed electrical stimulation to a buccal branch of the lingual nerve within the cheek region and receiving electrodes on a forehead region to deliver directed electrical stimulation to supraorbital branches within the forehead region;

FIG. 3 is a block diagram showing an electrical stimulator delivering at least one carrier wave to a modulator selectively modulating the at least one carrier wave to produce a modified electrical signal providing increased CSF flow through arterial vessels to the head and therefore increasing waste clearance;

FIG. 4 is a graph showing a magnitude of vasodilation/constriction of arterial vessels relative to a mean vessel diameter as a function of a time to return to baseline (TBL) at a given electrical stimulation frequency and duty cycle;

FIG. 5 is a timeline showing the electrical stimulation protocol for delivering layered low frequency modulated and low frequency unmodulated carrier waves during sleep and delivering the layered low frequency carrier waves with a high frequency unmodulated carrier wave in an alternating pattern during wakefulness; and

FIG. 6 is a graph showing a magnitude of CSF flow as a function of time during stimulation and no stimulation in order to “dial up” and “dial down” clearance in the brain for different drug profiles.

### DETAILED DESCRIPTION OF THE INVENTION

### Electrical Stimulation of Target Nerve Locations

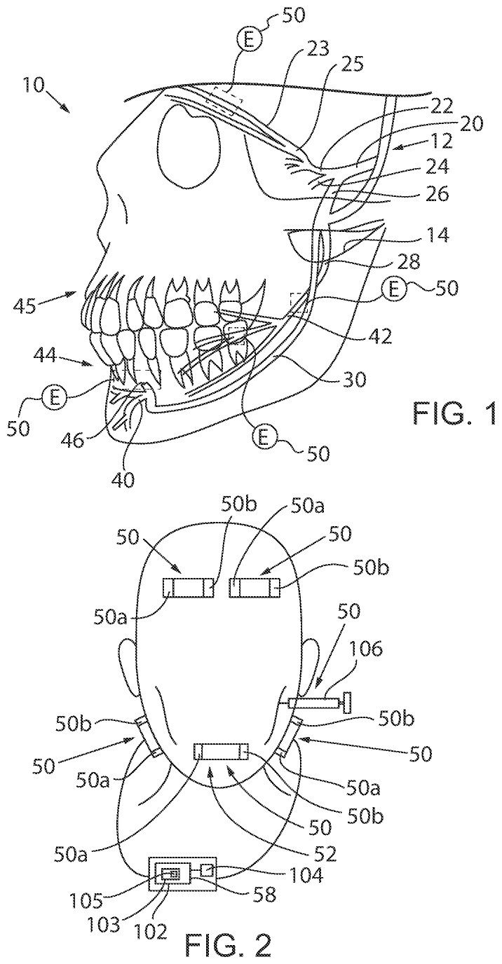

Referring to FIGS. 1 and 2, electrical stimulation of easily accessible neural inputs located outside of the brain and minimally invasive or non-invasive stimulation strategies can induce cardiovascular and respiratory changes, dilate arterial vessels, and increase the wall movement (change in the vessel diameters over time relative to a mean vessel diameter) of penetrating arterial vessels in the brain thus leading to increased clearance of misfolded proteins from the brain. Specifically, electrical stimulation of cranial nerves or local areas around the cranial nerves may selectively cause oscillations in pressure and dilation of arteries that help to improve waste clearance in the brain. In addition, stimulation of cranial nerves in the periphery leads to concomitant activity in the brain which can alter neuronal firing patterns through a process known as entrainment.

A typical human skull 10 supports a number of cranial nerves 12 emerging directly from the brain, located within the skull 10, and emerging out through cranial foramina 14, or holes, in the skull 10 to reach their final destinations on the exterior of the skull 10 and around the jaw and neck region. These cranial nerves 12 relay information between the brain and other parts of the body.

The trigeminal nerve 20 (fifth cranial nerve) is the largest of the cranial nerves 12 and provides sensation to the face and various motor functions such as biting and chewing functions. The trigeminal nerve 20 includes three major branches: the ophthalmic nerve (V1) 22, the maxillary nerve (V2) 24, and the mandibular nerve (V3) 26. The facial nerve (seventh nerve) has also been shown to be receptive to electrical stimulation.

The mandibular nerve (V3) 26 includes several sub-branches including the lingual nerve 28 and the inferior alveolar nerve 30 that have shown to be particularly receptive to electrical stimulation. The ophthalmic nerve (V1) 22 includes several sub-branches including the supraorbital nerves 23 that has shown to be particularly receptive to electrical stimulation.

Specifically, in a first embodiment, the mental branch nerves 40 are a sub-branch of the inferior alveolar nerve 30 and provides sensation to the front of the chin, lower lip, labial gingiva of the mandibular anterior teeth and the premolars. The buccal branch nerves of trigeminal nerves 42 are a sub-branch of the lingual nerve 28 that provides sensation to the cheek and the second and third molar teeth. The locations of the mental branch nerves 40 and buccal branch nerves of trigeminal nerves 42 in and around the lower jawbone 44 place the stimulation points or respective nerve endings 46 close to the outermost epidermis of the skin making it an ideal location for electrical stimulation where the distance and impedance between an electrode and the target nerve may be minimized. In this respect lower amounts of electrical energy may be needed to stimulate these sensory nerves compared to nerves located deeper within the skin and which may require more invasive procedures to stimulate the nerves.

Electrodes 50 placed at or proximate to the mental branch nerves 40 and buccal branch nerves of trigeminal nerves 42 allow for electrical stimulation of the respective nerves to therefore elicit a sustained response of the arterial vessels to dilate/constrict in a pulsating manner, as further discussed below. In one embodiment of the present invention, the electrodes are positioned over the mental foramen which may transmit electrical stimulation to the terminal branches of the inferior alveolar nerve and vessels of the mental artery.

Alternatively, in a second embodiment, the supraorbital nerves 23 are a sub-branch of the frontal nerve 25 which is a sub-branch of the ophthalmic nerve (V1) 22. The supraorbital nerves 23 provides sensation to the lateral forehead and the upper eyelid. The location of the supraorbital nerves 23 on the forehead places the stimulation points or respective nerve endings 46 close to the outermost epidermis of the skin making it an ideal location for electrical stimulation where the distance and impedance between an electrode 50 and the target nerve may be minimized. In this respect lower amounts of electrical energy may be needed to stimulate these sensory nerves compared to nerves located deeper within the skin and which may require more invasive procedures to stimulate the nerves.

Electrodes 50 placed at or proximate to the supraorbital nerves 23 allow for electrical stimulation of the nerves to therefore elicit a sustained response of the arterial vessels to dilate/constrict in a pulsating manner, as further discussed below. In one embodiment of the present invention, the electrodes 50 are positioned on the forehead which may transmit electrical stimulation to the terminal branches of the supraorbital nerves 23 and vessels of the frontal nerve 25.

Electrical stimulation of specific cranial nerves such as the branches of the trigeminal nerve, i.e., buccal branch nerves, lingual branch nerves, and supraorbital branch nerves, the facial nerve, vagus branch nerves, e.g., auricular vagus nerve, cervical nerve, sympathetic trunk/sympathetic ganglia, and sympathetic efferent branches create a more sustained vessel wall movement activity compared to stimulation of other cranial nerves. One possible explanation is that the facial and trigeminal nerves have direct sympathetic/parasympathetic innervation of the cerebral vasculature through several routes, including through the sphenopalatine ganglion (SPG) and greater superficial petrosal nerve that innervates the SPG, which are part of neural pathways that directly control the vasodilation/constriction of the cerebral arteries. As a result, the time course for dilation and constriction after a stimulation burst can be quicker than other cranial nerves because the response is quicker than inputs from the spinal cord which change peripheral sympathetic tone or peripheral inputs such as the sciatic nerve that change blood flow primarily through sensory activity mediated neurovascular coupling. Also, stimulation through pathways that change sympathetic/parasympathetic tone outside the brain dilate the peripheral vasculature outside of the brain. The change in blood flow in the brain is primarily in response to this change in peripheral blood flow to maintain perfusion (there are also occasionally indirect connections between the vagus and facial nerve in some subjects). As vagus nerve stimulation only indirectly influences blood flow in cerebral vasculature, it has a slower time constant between burst of stimulation for changes in flow to return to normal.

Electrical stimulation of specific nerve types may be desirable to elicit more CSF response but minimizes pain to the patient. For example, A-delta fibers with small diameter, i.e., 1 to 5 μm, serve to receive and transmit information primarily relating to acute pain (sharp, immediate, and relatively short-lasting) while A-beta fibers with medium diameter, i.e. 6 to 12 μm, and are afferent fibers associated with touch not pain. Therefore, stimulation of group A-beta nerve fibers may be preferred over group A-delta nerve fibers since they are less painful to innervate but provide similar CSF response outcomes.

### Electrical Stimulation Modalities

Referring still to FIGS. 1 and 2, in one embodiment of the present invention, the electrodes 50 may be a part of an intraoral device 52 placed in the mouth, such as a mouthguard, dental filling or dental implant, to electrically stimulate the inferior alveolar nerve 30 and mental branch nerves 40 located near or around the lower jawbone 44 region. The intraoral device 52 may be used to conveniently and consistently position the electrodes 50 at specific locations in the mouth to provide electrical stimulation 72 to target nerves. For example, the electrodes 50 may be placed in close proximity to the mental foramen and/or the inferior alveolar nerve 30 and mental branch nerves 40.

In one embodiment of the present invention, the intraoral device 52 is a mouthpiece receivable into a patient's mouth and configured to carry the electrical circuitry of an electrical stimulator 58, and generally including a processor 102 and battery power supply 104 (for example, including rechargeable lead acid or lithium ion batteries) for delivery of electrical stimulation 72 to the electrodes 50 within the mouth. It is understood that the electrical stimulator 58 may be positioned outside of the mouth and communicate remotely with a controller and electrodes 50 of the intraoral device 52.

The battery power supply 104 may provide power to an electrical stimulator 58 in a manner which allows for current delivery to the electrodes 50. It is understood that an external power supply may also be used to deliver power to the electrical stimulator 58 in addition to or instead of the battery power supply 104.

A desired position of the electrodes 50 on the walls of the intraoral device 52 may be determined by a location of the mental branch nerves 40 and nerve endings 46 in the mouth. In this respect, medical imaging may be used to help locate the position of the mental branch nerves in the mouth and to aid in placement of the electrodes 50 on the intraoral device 52.

The electrodes 50 may include a stimulating cathode electrode 50a placed close to the desired stimulation site, for example, near the nerve endings 46 of the mental branch nerves 40. An anode electrode 50b may then be placed proximal to the cathode electrode 50a with respect to the nerve endings 46. In this respect, the electrical current flows from the anode electrode 50b to the cathode electrode 50a so that the nerve endings 46 receives the stimulus and propagates an action potential upstream through the mental branch nerves 40. The cathode electrode 50a and anode electrodes 50b are spaced apart along an axis generally parallel to the course of the mental branch nerves 40.

Medical imaging may be used to facilitate precise placement of the cathode electrode close to the nerve ending and the precise placement of the anode electrode 50b upstream from the cathode electrode 50a and proximal to the nerve ending 46. The cathode electrode 50a may be placed less than 2 cm or less than 1 cm from the mental branch nerve ending 46 and the anode electrode 50b may be placed less than 3 cm or less than 2 cm upstream from the cathode electrode 50a along the mental branch nerves 40.

It is understood that the pair of communicating electrodes, i.e., the cathode electrode and anode electrode 50b, may stimulate at least one of the left and right side mental branch nerves 40 of the mouth and the intraoral device 52 may include more than one pair of anode and cathode electrodes 50a, 50b to stimulate both the left and right mental branch nerves 40 of the left and right sides of the mouth. It is also understood that more than two pairs of anode and cathode electrodes 50a, 50b may be carried by the intraoral device 52 to stimulate various areas along the mental branch nerves 40. In some embodiments, changing the phase of the modulating signal 70 to different mental branch nerves 40 may be used to enhance the stimulation of the mental branch nerves 40 by timing the delivery of the electrical stimulation to the multiple pairs of electrodes. In one embodiment, electrical simulation may be rotated across several electrodes placed across the target nerve, to maximize activation of that nerve without activating nearby nerves responsible for unwanted side effects.

In an alternative embodiment of the present invention, the electrodes 50 may be surface electrodes placed on an outer surface of the cheek, or subcutaneous electrodes inserted under the skin in the cheek region to electrically stimulate the buccal branch nerves of trigeminal nerves 42 located in an upper jawbone 45 region and the facial nerves below the cheek.

For example, the electrodes 50 are surface electrode pads placed externally on the patient's cheeks overlying the buccal branch nerves 26 to stimulate the buccal branch nerves 26 and/or facial nerves such as ophthalmic nerve (V1) 22 and mandibular nerve (V3) 26. The electrodes 50 may include a cathode electrode 50a and anode electrode 50b placed externally on the same cheek. Current flow between the anode and cathode electrodes 50a, 50b may provide electrical stimulation of the target nerve. A second set of anode and cathode electrodes 50a, 50b may be placed on the opposite cheek to stimulate the buccal branch nerves 26 and/or facial nerves of the opposite cheek. In one embodiment, non-invasive stimulation of the ophthalmic nerve (V1) 22 and mandibular nerve (V3) 26 has been found to cause increases in blood flow as seen in MRI 4D-flow imaging comparable to clinical methods of increasing blood flow using the drug dobutamine which increases heart rate contractility.

In another example, the electrodes 50 may be subcutaneous electrodes inserted beneath the epidermis within the patient's cheeks to stimulate the buccal branch nerves 26. The subcutaneous electrodes may be injectable electrodes 50 such as liquid metal electrodes injectable using a syringe 106 and withdrawable from the skin. The injectable electrodes 50 may include a cathode electrode 50a and anode electrode 50b placed under the skin. Current flow between the anode and cathode electrodes 50a, 50b may provide electrical stimulation of the target nerve. The injectable electrodes 50 may be injected into one or both cheeks to stimulate the buccal branch nerves 26.

In an alternative embodiment of the present invention, the electrodes 50 may be surface electrodes placed on an outer surface of the forehead above the eye, or subcutaneous electrodes inserted, e.g., by injection, under the skin in the forehead region, to electrically stimulate the supraorbital branches of the trigeminal nerve or trigeminal nerve fibers in a similar manner as described above with respect to electrodes 50 placed in the cheek region to electrically stimulate the buccal branch nerves of trigeminal nerves 42 located in an upper jawbone 45 region and the facial nerves below the cheek. Electrode placement can be improved by palpating the foramen to isolate and target the trigeminal nerve as it leaves the foramen above the orbit where it is most superficial and thus avoiding unwanted off-target activation.

In an alternative embodiment of the present invention, the electrodes 50 may be surface electrodes providing non-invasive stimulation of the auricular vagus at the cymba conchae/tragus. The electrodes 50 may be part of an earbud or headband supporting the electrodes 50 over the tragus.

In some embodiments, the stimulating electrodes 50 may be used to record electrophysiological signals in the brain, e.g., to detect changes in low frequency power brain waves that propagate outside the calvarium. The stimulating electrodes 50 may also be used to pick up other physiological signals of the patient such as heart rate and heart rate variability. Alternatively, separate electrodes from the stimulating electrodes 50 may be placed on the patient to record the electrophysiological signals in the brain and other physiological signals.

The electrode 50 placement and intraoral device 52 may be as described in U.S. Pat. No. 11,395,914, titled “Penetration of Cerebral Spinal Fluid into the Brain Parenchyma using Temporally Pattered Neuromodulation,” filed Jul. 22, 2020, assigned to the present applicant and hereby incorporated by reference.

Other methods for modulating glymphatic clearance described herein may include any appropriate form of stimulation of the nerves. Examples of electrical stimulation modalities that may be used as described herein include, without limitation, peripheral nerve stimulation (e.g., vagus nerve stimulation and/or carotid sinus nerve stimulation), transcranial direct or alternate current stimulation (tDCS/tACS), deep brain stimulation (DBS), cortical stimulation, spinal cord stimulation (SCS), transcranial/transdermal magnetic stimulation (TMS), focused ultrasound, infrared stimulation, optogenetic activation, genetic modification to enhance sensitivity and specificity of the nerve to stimulation with a light source (optogenetics), and use of intravascular electrodes.

### Synergistic Electrical Stimulation Protocols

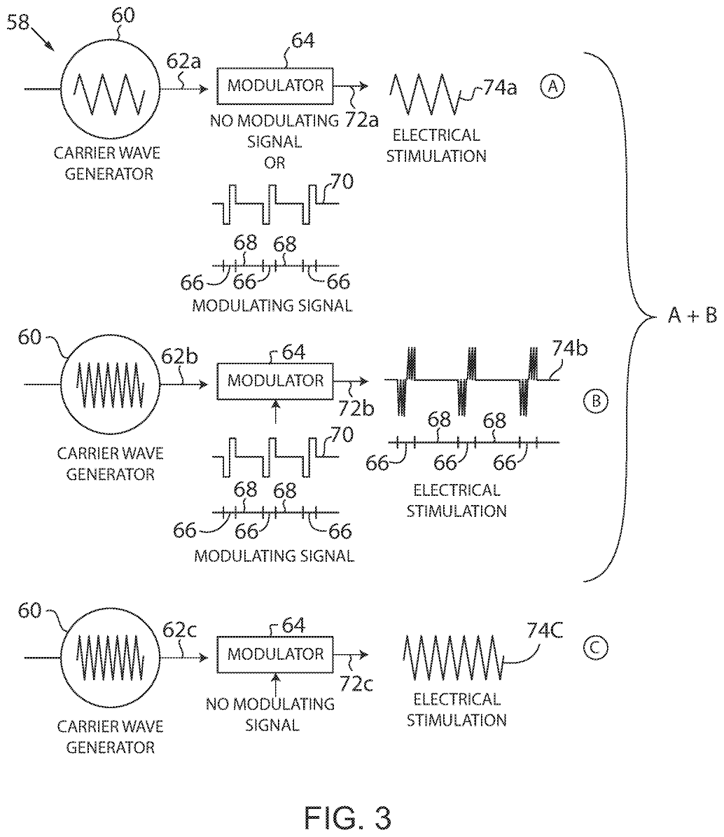

Referring now to FIGS. 2 and 3, a layered electrical stimulation protocol that (1) optimizes AQP4 mediated fluid exchange (i.e., fluid and molecular movement), (2) uses temporal patterning and stimulation parameters maximizing the cerebral vessel wall movement, (3) promotes phagocytic phenotype in glial cells to break down waste proteins, and (4) reduces neural activity to decrease fluid within the neuron, thereby increasing fluid in the extracellular space to reduce the resistance of the paranchymal pathway to fluid flow promoting clearance, are used to synergistically increase the CSF flow rate (i.e., flow speed) to the periarterial spaces to maximize waste clearance from the brain. The vessel wall movement may be defined as a change in the vessel diameter over time relative to a mean vessel diameter.

Electrical stimulation of the target nerves may be accomplished using an electrical stimulator 58 such as those commercially available from Tucker-Davis Technologies of Alachua, Florida or A-M Systems of Sequim, Washington. The electrical stimulator 58 may include a carrier wave generator 60 and a processor 102 being an electronic computer having a self-contained nonvolatile memory 103 holding an operating program 105 and necessary storage variables as will be described below. The nonvolatile memory 103 may comprise, for example, flash memory and/or read only memory, or other similar nonvolatile memory as context requires, which may store data values to be retained even in the absence of electrical power. The processor 102 may be a STM32 Nulceo board or PIC microcontroller as known in the art.

The processor 102 provides various inputs and output lines communicating, for example, with one or more stored programs 105 stored in non-transitory memory 103 and the carrier wave generator 60 to generate one or more carrier waves 62 at a carrier frequency and amplitude. The one or more carrier waves 62 are delivered to a modulator 64 modulating the one or more carrier waves 62 amplitude according to a modulating signal 70.

Referring specifically to FIG. 3, in one embodiment of the present invention, the processor 102 may communicate with one or more stored programs 105 stored in a non-transitory memory 103 and the carrier wave generator 60 to generate first, second, and third carrier waves 62a, 62b, 62c where the carrier waves 62a, 62b, 62c are delivered to the modulator 64, also communicating with the processor 102 and modulating amplitude of the second carrier waves 62b according to the modulating signal 70. In some embodiments, the first and third carrier waves 62a, 62c passes through the modulator 64 without modulation or alternatively may bypass the modulator 64 entirely if it is known that no modulation is needed to be applied to the first and third carrier wave 62a, 62c.

A first carrier wave 62a is delivered continuously at a low frequency between 0.1 Hz and 4.5 Hz and preferably centered around <1 Hz. The low frequency is desirably consistent with a frequency of electroencephalogram (EEG) brain activity during slow-wave sleep. Slow-wave sleep is phase 3 sleep and the deepest stage of non-rapid eye movement sleep. The slow-wave sleep is characterized by delta waves (between 0.5 Hz and 4.5 Hz). The first carrier wave 62a may have a current amplitude of less than 1000 microamps for invasive stimulation and less than 40 milliamps for non-invasive stimulation and a voltage controlled to achieve this current per current control known in the art.

The first carrier wave 62a will pass through the modulator 64 without amplitude modification or without significant amplitude modification, or alternatively, will bypass the modulator 64 entirely to provide a first continuously electrical stimulation 72a output that has an amplitude and continuous duty cycle that is the same or similar to the first carrier wave 62a. Therefore, the first carrier wave 62a may be delivered continuously during and after a patient's sleep state to encourage opening of the AQP4 channels.

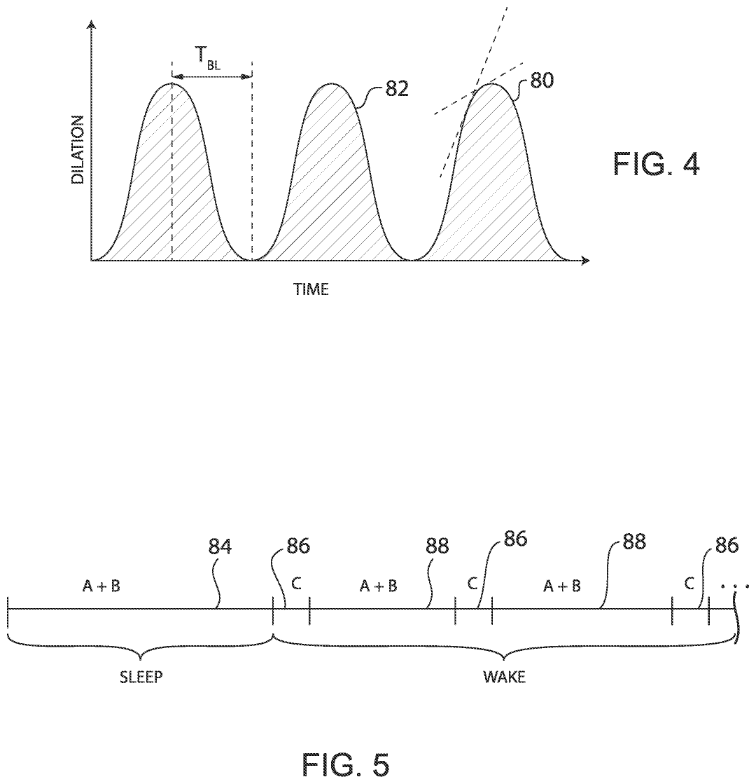

In some embodiments, the first carrier wave 62a is modulated and will pass through a modulator 64 to provide a temporal pattern of low frequency pulses 74a in the first electrical stimulation 72a output. In a first modulation period 66, e.g., “ON” pulse interval, there is minimal to no modification of the first carrier wave 62a and the first carrier wave 62a is allowed to pass without modifying or substantially modifying the amplitude of the signal (i.e., stimulation “ON” state) or has a comparatively higher amplitude, and in a second modulation period 68, e.g., “OFF” pulse interval, the modulator 64 will modify the first carrier wave 62a to reduce the electrical stimulation amplitude to substantially zero (i.e., stimulation “OFF” state) or has a comparatively lower amplitude. In this case, the modulating signal 70 may be a discontinuous waveform such as a biphasic pulse or square wave. As is understood in the art, signal modulation by the modulator 64 may provide an envelope of the peaks of the first carrier wave 62a, the latter being of much higher frequency than the modulating signal 70. Although the modulating signal is shown as a square wave in FIG. 3, the modulating signal 70 may also be a smooth curve as shown in FIG. 4. It is understood that during modulation, the modulator 64 may modify the first carrier wave 62a to an amplitude that differs between the first modulation period 66 and the second modulation period 68. For example, the amplitude of the first carrier wave 62a may be greater during the first modulation period 66 as compared to the second modulation period 68 and vice versa.

Referring briefly to FIG. 4, the stimulation parameters of the first carrier wave 62a may be empirically set to maximize the opening of AQP4 channels. While the inventors do not wish to be bound by a particular theory, it is believed that low delta frequency stimulation (e.g., to 4 Hz) delivered continuously during patient sleep or during patient wakeful state increases the fluid flux across AQP4 channels in the astrocytic endfeet surrounding descending arterioles in the brain. During the patient wakeful state, the low frequency stimulation may compensate for lowered fluid flux of AQP4 channels when slow-wave sleep frequencies are not naturally occurring.

This first carrier wave 62a set point may be established, for example, by monitoring a set of patients being scanned in a computed tomography (CT) scanner or magnetic resonance imaging (MM) scanner with contrast media during or immediately following delivery of the first electrical stimulation 72a to detect opening of AQP4 channels and CSF/ISF flow and adjusting the amplitude and frequency of the first carrier wave 62b to maximize the area 80 beneath the CSF/ISF flow curve 82. It is understood that other biological measurements of autonomic nerve activity may be used to determine effectiveness of CSF/ISF clearance including blood pressure, galvanic skin response, heart rate, respiration variability, fraction anisotropy, and presence of certain biomarkers in the patient's blood, CSF, or saliva. These settings may then be used generally for all patients or may be optimized for particular patient classes such as by age, height and weight, sex, and genetic predispositions to specific diseases.

Referring again to FIGS. 2 and 3, a second carrier wave 62b is delivered continuously at a higher frequency between 20 Hz and 75 Hz and between 20 Hz and 40 Hz and between 10 Hz to 15 Hz and preferably centered around 30 Hz. The second carrier wave 62b may have a current amplitude of less than 1000 microamps for invasive stimulation and less than 40 milliamps for non-invasive stimulation and a voltage controlled to achieve this current per current control known in the art.

The second carrier wave 62b will pass through the modulator 64 to provide a temporal pattern of high frequency pulses 74b in the second electrical stimulation 72b output. In a first modulation period 66 or “ON” pulse interval, there is no modification of the second carrier wave 62b and the second carrier wave 62b is allowed to pass without modifying the amplitude of the signal (i.e., stimulation “ON” state), and in a second modulation period 68 or “OFF” pulse interval, the modulator 64 will modify the second carrier wave 62b to reduce the electrical stimulation amplitude to substantially zero (i.e., stimulation “OFF” state). In this case, the modulating signal 70 may be a discontinuous waveform such as a biphasic pulse or square wave. As is understood in the art, signal modulation by the modulator 64 may provide an envelope of the peaks of the second carrier wave 62b, the latter being of much higher frequency than the modulating signal 70. Although the modulating signal 70 is shown as a square wave in FIG. 3, the modulating signal 70 may also be a smooth curve as shown in FIG. 4.

Referring briefly to FIG. 4, the stimulation parameters of the second carrier wave 62b and the modulating signal 70 may be empirically set to maximize arterial wall movement. While the inventors do not wish to be bound by a particular theory, it is believed that the accommodation or acclamation of the tissue to the stimulation effectively limits the clearance when continuous stimulation is provided as understood in the prior art. By interleaving higher frequency stimulation (of “ON” pulse interval 66) with periods of rest (of “OFF” pulse interval 68), vasoconstrictive/dilative recovery processes may be accommodated to maximize pulsatile motion and allow greater clearance in the long run. It has been found that continuous “ON” pulse intervals for long periods of time, e.g., 5 Hz or 10 Hz for greater or equal to 30 seconds, without periods of rest (“OFF” pulse interval) habituates blood flow changes (i.e., blood flow changes go back to baseline) and do not enable the desired outcomes in cerebral blood flow.

Ideally, the electrical stimulation parameters of the modulating signal 70 will provide a relaxation time (of “OFF” pulse interval 68) defined by the second carrier wave 62b being in an “OFF” state that is no less than the time to return to baseline (TBL) measured after brief periods of stimulation (of “ON” pulse interval 66) defined by the second carrier wave 62b being in an “ON” state. The ratio of the pulse duration of the “ON” stimulation to the total period of the ON/OFF waveform may be referred to as the “duty cycle” of the modulating signal 70.

This second carrier wave 62b set point may be established, for example, by monitoring a set of patients being scanned in a computed tomography (CT) scanner or magnetic resonance imaging (MRI) scanner with contrast media during or immediately following delivery of a second electrical stimulation 72b to detect vessel wall movement and CSF/ISF flow and adjusting the amplitude and frequency of the second carrier wave 62b and temporal pattern or “duty cycle” of the modulating signal 70, i.e., stimulation time (stimulation “ON” state) and relaxation time (stimulation “OFF” state), to maximize the area 80 beneath the CSF/ISF flow curve 82. Thus, the dilation/constriction of arterial vessels at various modulating signal frequencies may be compared to maximize the area 80 under the curve 82 of FIG. 4, for example, slower, large amplitude changes in clearance (produced by prolonged carrier frequency stimulation) may be compared with faster, smaller amplitude changes in clearance (produced by shortened carrier frequency stimulation) to provide the greatest increases in CSF flow over time in the perivascular space. Similar comparisons may be done with respect to the spacing between stimulations provided by the relaxation period, i.e., the “duty cycle”.

It is understood that other biological measurements of autonomic nerve activity may be used to determine effectiveness of CSF/ISF clearance including blood pressure, galvanic skin response, heart rate, respiration variability, fraction anisotropy, and presence of certain biomarkers in the patient's blood or saliva. These settings may then be used generally for all patients or may be optimized for particular patient classes such as by age, height and weight, sex, and genetic predispositions to specific diseases.

Referring again to FIGS. 2 and 3, preferably during patient sleep or periods of slow-wave sleep, the first carrier wave 62a and second carrier wave 62b are overlaid or delivered simultaneously to provide a synergistic effect of greater consistent opening of AQP4 water channels found in the astrocytic endfeet and movement of CSF flow in the parenchymal extracellular space through increased vessel wall movement, to create larger than expected CSF-ISF fluxes into and out of the brain or spinal cord as part of the CSF-ISF exchange.

Referring briefly to FIG. 4, while the stimulation parameters of the first electrical stimulation 72a and second electrical stimulation 72b may be empirically set separately, as described above, the first and second carrier waves 62a, 62b and modulating signal 70 may additionally or alternatively be empirically set when observed together so as to establish maximum synergistic effect of the first and second electrical stimulation 72a, 72b and modulating signal 70 protocols to maximize the clearance of CSF. In this respect, the delivery of the stimulation parameters of the first carrier wave 62a and second carrier wave 62b as described above provides unexpected effects that are greater than the effects of each carrier wave 62a, 62b delivered separately and combined, which may be expected by the prior art understanding.

Referring to FIGS. 2, 3 and 5, the overlaid, simultaneously delivered first carrier wave 62a and second carrier wave 62b will be delivered continuously during patient sleep but during patient wakeful state, may be periodically replaced by a third carrier wave 62c. In some embodiments, the third carrier wave 62c may be delivered sporadically during sleep, e.g., patient mid-sleep or in between sleep cycles.

The third carrier wave 62c is delivered continuously at high frequency, gamma rhythms between 25 and 140 Hz and centered around 40 Hz. The third carrier wave 62c may have a current amplitude of less than 1000 microamps for invasive stimulation and less than 40 milliamps for non-invasive stimulation and a voltage controlled to achieve this current per current control known in the art.

The third carrier wave 62c will pass through the modulator 64 without modification or without substantial modification or alternatively will bypass the modulator 64. The third carrier wave 62c may be delivered continuously for, e.g., periods of 15 minutes to 60 minutes, between periods of administering the first carrier wave 62a and second carrier wave 62b simultaneously.

Referring briefly to FIG. 4, the stimulation parameters of the third carrier wave 62c may be empirically set to promote a more phagocytic phenotype in glial cells to help break down waste biomolecules and misfolded proteins for subsequent clearance. While the inventors do not wish to be bound by a particular theory, it is believed that glial cells play a role in neurodegenerative conditions. Among glial cells, microglia and astrocytes play phagocytic roles by engulfing synapses, apoptotic cells, cell debris, and released toxic proteins. By administration of electrical stimulation at gamma frequency (i.e., 20-50 Hz) for longer durations (e.g., 15 minutes to 60 minutes), the expression of pro-phagocytic genes is increased, which promotes active phagocytic phenotypes states in glial cells. It is understood that flickering sound and light stimulation at gamma frequency (i.e., 20-50 Hz) may also enhance phagocytic states in glial cells and may be concurrently administered with electrical stimulation.

This third carrier wave 62c set point may be established, for example, by monitoring a set of patients being scanned in a computed tomography (CT) scanner, positron emission tomography (PET) scanner, or magnetic resonance imaging (MRI) scanner with contrast media during or immediately following delivery of a third electrical stimulation 72c to detect CSF/ISF flow, arterial dilation, presence and concentration of exogenously delivered markers of misfolded proteins/aggregates, movement of metabolic waste within CSF including misfolded proteins. and adjusting the stimulation amplitude and frequency to maximize the area 80 beneath the CSF/ISF flow curve 82. It is understood that other biological measurements of autonomic nerve activity may be used to determine effectiveness of CSF/ISF clearance including blood pressure, galvanic skin response, heart rate, respiration variability, fraction anisotropy, and presence of certain biomarkers in the patient's blood or saliva. These settings may then be used generally for all patients or may be optimized for particular patient classes such as by age, height and weight, sex, and genetic predispositions to specific diseases.

While the stimulation parameters of the third carrier wave 62c may be empirically set independently as described above, the third carrier wave 62c may additionally or alternatively be empirically set when administered with the first carrier wave 62a, second carrier wave 62b, and modulating signal 70 so as to establish maximum synergistic effect of the three electrical stimulation protocols to maximize the clearance of CSF. In this respect, the delivery of the stimulation parameters of the first carrier wave 62a and second carrier wave 62b together with the third carrier wave 62c as described provides unexpected effects that are greater than the effects of each carrier wave 62a, 62b, 62c delivered separately and combines which may be expected by the prior art understanding.

It is understood that in some embodiments, the third carrier wave 62c may be administered prior to the administration of the first carrier wave 62a, second carrier wave 62b, and modulating signal 70, and as an initial step in the stimulation protocol in order to induce phagocytosis prior to increasing vessel wall movement and CSF clearance with the first carrier wave 62a, second carrier wave 62b.

Referring again to FIGS. 1 and 2, the electrical stimulator 58 may communicate with the electrodes 50 of, for example, an intraoral device 52 to deliver the electrical pulses 74a, 74b, 74c of the electrical stimulation 72a, 72b, 72c, respectively, to the electrodes 50. In one embodiment, the electrical stimulator 58 may be external to the intraoral device 52 and communicate wirelessly with the electrodes 50 on the intraoral device 52. In an alternative embodiment, the electrical stimulator 58 may be external to the intraoral device 52 or incorporated or molded onto the intraoral device 52 to communicate with the electrodes 50 on the intraoral device 52 via a wired connection.

In an alternative embodiment, the electrical stimulator 58 may also communicate with electrodes 50 which are surface electrodes or subcutaneous electrodes or other electrical stimulation modalities as previously described.

It is contemplated that there may be multiple electrodes 50 positioned at different locations on the patient to stimulate different target nerves. Each of the multiple electrodes 50 may deliver different electrical pulses 74a, 74b, 74c and at different times in order to optimize maximum synergistic effect of the three electrical stimulation protocols to maximize the clearance of CSF. This may be desired based on different target nerves responding differently to the electrical stimulation 72a, 72b, 72c and the respective frequencies. For example, the first carrier wave 62a and second carrier wave 62b may be delivered to a first target nerve that responds well to increasing arterial wall movement and the third carrier wave 62c may be delivered to a second target nerve that responds well to inducing phagocytosis. Although the inventors do not wish to be bound by a particular theory, it is believed that certain target nerves are better at modulating arterial wall movement while others are better at modulating astrogliotic activity.

The following is an exemplary embodiment of modulating signal frequencies and temporal patterning of the three electrical stimulation protocols of the first, second and third carrier waves 62a, 62b, 62c, delivered synergistically to optimize CSF clearance.

### Example 1: Synergistic Stimulation Protocol

Referring to FIGS. 3 and 5, the first carrier wave 62a may be a single frequency waveform (e.g., a cathodic leading, biphasic sine wave) with the frequency of the first carrier wave 62a being less than 4.5 Hz, and between 0.1 and 4.5 hertz and preferably between 1 and 3 hertz, and the preferred range centered around 2 Hz. The first carrier wave 62a may be at an amplitude of 800 μA and has a pulse width of 200 μs. Although the inventors do not wish to be bound by a particular theory, the stimulation intensity was found to have an inverted U-function with cortical plasticity with medium range amplitudes (400-800 μA) exhibiting optimal effects compared to low (<400 μA) and high (>1.2 mA) amplitudes. The first carrier wave 62b may be delivered without modulation to provide the first electrical stimulation 72a that is continuous and substantially the same amplitude as the first carrier wave 62a.

In some embodiments, the first carrier wave 62b is delivered with modulation and the modulating signal 70 of the modulator 64 applied to the first carrier wave 62a may be a single frequency, monophasic signal such as a sine wave creating “bursts” of electrical stimulation. The frequency of the modulating signal 70 is preferably between 30 to 40 hertz. The modulating signal 70 may provide the first electrical stimulation 72a with electrical pulses 74a with a 1% to 20% duty cycle and the electrical pulses 74a having an “ON” pulse interval 66 between 1 second and 2 seconds, and “OFF” pulse intervals 68 between 5 seconds and 100 seconds.

The second carrier wave 62b may be a single frequency waveform (e.g., a cathodic leading, biphasic sine wave) with the frequency of the second carrier wave 62b being less than hertz, and between 20 hertz and 60 hertz, and preferably between 25 hertz and 55 hertz, with the preferred range centered around 30 hertz or 50 hertz. At higher frequencies (75 Hz or above), habituation occurs before peak flow change is obtained, causing a weaker effective pulse therefore the frequency is preferably below 75 Hz.

The second carrier wave 62b may be at an amplitude of 800 μA and has a pulse width of 200 μs. Although the inventors do not wish to be bound by a particular theory, the stimulation intensity was found to have an inverted U-function with cortical plasticity with medium range amplitudes (400-800 μA) exhibiting optimal effects compared to low (<400 μA) and high (>1.2 mA) amplitudes.

The modulating signal 70 of the modulator 64 applied to the second carrier wave 62b may be a single frequency, monophasic signal such as a sine wave creating “bursts” of electrical stimulation. The frequency of the modulating signal 70 is preferably between 0.5 hertz and 0.1 hertz. The modulating signal 70 may provide the second electrical stimulation 72b with electrical pulses 74b with a 5% to 50% duty cycle and the electrical pulses 74b having an “ON” pulse interval 66 between 1 second and 240 seconds, and “OFF” pulse intervals 68 between 1 second and 300 seconds. Although the inventors do not wish to be bound by a particular theory, the introduction of a longer relaxation period where there is no stimulation (“OFF” pulse intervals 68) counterintuitively increases total clearance because continuous stimulation habituates and is frequency dependent, e.g., a) 5 repetitions of buccal branch stimulation for 30 seconds at 10 Hz (200 microsecond pulse width, 1.5 mA, 30 seconds starting at −10 seconds) and b) 5 repetitions of buccal branch stimulation for 30 seconds at 5 Hz (200 microsecond pulse width, 1.5 mA, 30 seconds starting at −10 seconds) both habituate change in blood flow from baseline, i.e., blood flow changes go back to baseline, but the 10 Hz habituates faster. The specific temporal patterning of relaxation period and pulse intervals applied to specific target nerves as providing increased CSF flow is described with respect to Examples 2-5 below.

The third carrier wave 62c may be a single frequency waveform (e.g., a cathodic leading, biphasic sine wave) with the frequency of the third carrier wave 62c greater than 25 Hz and between 25 and 140 Hz and preferably between 25 and 60 Hz and the preferred range centered around 40 Hz. The third carrier wave 62c may be at an amplitude of 800 μA and has a pulse width of 200 μs. The third carrier wave 62c may be delivered continuously without modulation to provide the third electrical stimulation 72c with an amplitude that is substantially the same as the third carrier wave 62c.

The first and second electrical stimulation 72a, 72b may be delivered simultaneously and continuously for a first period 84 that is consistent with a patient's sleep duration or for at least one non-rapid eye movement (NREM) sleep cycle. The first period 84 may be at least 30 minutes or at least 1 hour and at least 2 hours and at least 3 hours and at least 4 hours and at least 5 hours and at least 6 hours and at least 7 hours and at least 8 hours and between 1 to 8 hours in duration consistent with at least one NREM sleep cycle and during periods of deep wave sleep.

The third electrical stimulation 72c may replace the first and second electrical stimulation 72a, 72b delivered simultaneously, to be delivered periodically (regular occurring intervals) and continuously for a second period 86 following patient sleep. It is understood that due to the high frequency of the third carrier wave 62c, delivery of the third electrical stimulation 72c may disrupt a patient's sleep, therefore, it is preferably delivered during a wakeful state of the patient. In some embodiments, a period of the third electrical stimulation 72c may be delivered mid-sleep or sometime during the sleep period to help promote clearance during sleep state.

Referring specifically to FIG. 5, the third electrical stimulation 72c is delivered for a second period 86 which may be at least 15 minutes and at least 30 minutes and at least 45 minutes and at least 1 hour duration and less than 30 minutes and less than 45 minutes and less than 1 hour and may be a period of 15 minutes to 60 minutes. Following the delivery of the third electrical stimulation 72c, delivery of the first and second electrical stimulation 72a, 72b may resume for a third period 88 during a patient's wakeful state until being interrupted by the subsequent repeated second period 86 of the third electrical stimulation 72c. The second period 86 and third periods 88 occur during the wakeful state of the patient and may repeat for a duration of at least 1 hour and at least 2 hours and at least 3 hours and at least 4 hours and at least hours and at least 6 hours and at least 7 hours and at least 8 hours and at least 9 hours and at least 10 hours and at least 11 hours and at least 12 hours and between 1 to 16 hours and consistent with a desired length of treatment during the patient's wakeful state.

The delivery of the third electrical stimulation 72c will be delivered periodically (at regular occurring intervals), for example, every 30 minutes, every 60 minutes, every 90 minutes, every 120 minutes, or every 30 to 120 minutes in changing time intervals that may increase over time. The second period of time 86 may be repeated at least twice or at least three times or at least four times or at least five times or continuously during the patient's wakeful state and during the concurrent but separate administration of the first and second electrical stimulation 72a, 72b during the patient's wakeful state.

In some embodiments, the duration of the third electrical stimulation 72c of the second period of time 86 may be reduced over time for subsequent deliveries of the third electrical stimulation 72c, for example, when less waste proteins need to be cleared after subsequent deliveries of the third electrical stimulation 72c. Also, the amount of time between the third electrical stimulation 72c may be increased over time for subsequent deliveries of the third electrical stimulation 72, for similar reasons.

The delivery of the third electrical stimulation 72c enhances clearance of waste proteins between cycles of increased CSF-ISF fluxes into and out of the brain created by the first and second electrical stimulation 72a, 72b and thus enabling more clearance to occur.

The multiple electrical stimulations 72a, 72b, 72c are delivered to the target nerve of the patient via electrodes 50. It is understood that the electrical stimulation of the present invention may be applied to target nerves identified as providing increased CSF flow, for example, facial nerves, trigeminal nerves, sphenopalatine ganglia, carotid sinus nerve, and baroreceptor, sciatic nerve and peripheral nerve, vagus nerve (e.g., auricular vagus nerve), cervical nerve, sympathetic trunk/sympathetic ganglia, and sympathetic efferent branches, as previously described above.

The following are exemplary embodiments of stimulation protocols for modulating the second carrier wave 62b signal frequencies for specific target nerves optimized to create a full pulse without attenuating the peak flow response but accelerating the return to baseline. The first carrier wave 62a and third carrier wave 62c may be delivered to produce electrical stimulation 72a, 72c as previously described above.

### Example 2: Temporal Pattern for Vagus Nerve (Animal Model)

The first carrier wave 62a and third carrier wave 62c may be delivered to produce electrical stimulation 72a, 72c as described above with respect to Example 1.

The second carrier wave 62b may be a single frequency waveform (e.g., a sine wave) where the frequency of the carrier wave 62 may be less than 75 hertz, and between 20 hertz and hertz and preferably between 25 hertz and 50 hertz, with the preferred range centered around hertz.

The modulating signal 70 may provide second electrical stimulation 72b with electrical pulses 74b with a 5% to 15% duty cycle and preferably 10% duty cycle and electrical pulses 74b having “ON” pulse interval 66 between 1 second and 60 seconds, and preferably 30 seconds, and “OFF” pulse intervals 68 between 200 second and 300 seconds, and preferably 270 seconds between pulses.

### Example 3: Temporal Pattern for Facial Nerves, Trigeminal Nerves, and Sphenopalatine Ganglia

The first carrier wave 62a and third carrier wave 62c may be delivered to produce electrical stimulation 72a, 72c as described above with respect to Example 1.

The second carrier wave 62b may be a single frequency waveform (e.g., a sine wave) with the frequency of the carrier wave 62b less than 75 hertz, and between 20 hertz and 60 hertz and preferably between 25 hertz and 55 hertz, with the preferred range centered around 50 hertz.

The modulating signal 70 may provide second electrical stimulation 72b with electrical pulses 74b with a 40% to 60% duty cycle and preferably 50% duty cycle and electrical pulses 74b having “ON” pulse interval 66 between 1 second and 10 seconds, and preferably 5 seconds, and “OFF” pulse intervals 68 between 1 second and 10 seconds, and preferably 5 seconds between pulses.

### Example 4: Temporal Pattern for Vagus Nerve, Carotid Sinus Nerve, and Baroreceptor

The first carrier wave 62a and third carrier wave 62c may be delivered to produce electrical stimulation 72a, 72c as described above with respect to Example 1.

The second carrier wave 62b may be a single frequency waveform (e.g., a sine wave) where the frequency of the carrier wave 62 may be less than 75 hertz, and between 20 hertz and hertz and preferably between 25 hertz and 50 hertz, with the preferred range centered around hertz.

The modulating signal 70 may provide second electrical stimulation 72b with electrical pulses 74b with a 30% to 35% duty cycle and preferably 33% duty cycle and the electrical pulses 74b having “ON” pulse interval 66 between 15 seconds and 60 seconds, and preferably 30 seconds, and “OFF” pulse intervals 68 between 30 seconds and 120 seconds between pulses, and preferably 60 seconds between pulses.

### Example 5: Temporal Pattern for Sciatic Nerve and Peripheral Nerve

The first carrier wave 62a and third carrier wave 62c may be delivered to produce electrical stimulation 72a, 72c as described above with respect to Example 1.

The second carrier wave 62b may be a single frequency waveform (e.g. b a sine wave) with the frequency of the carrier wave 62b may be less than 75 hertz, and between 20 hertz and hertz and preferably between 25 hertz and 55 hertz, with the preferred range centered around hertz.

The modulating signal 70 may provide second electrical stimulation 72b with electrical pulses 74b with a 35% to 40% duty cycle and preferably 37.5% duty cycle and the electrical pulses 74 having an “ON” time interval 66 between 60 seconds and 240 seconds, and preferably 180 seconds, and “OFF” pulse intervals 68 between 240 seconds and 360 seconds between pulses, and preferably 300 second pulses.

### Real-Time Dial to Modulate Drug Delivery Profiles in the Brain

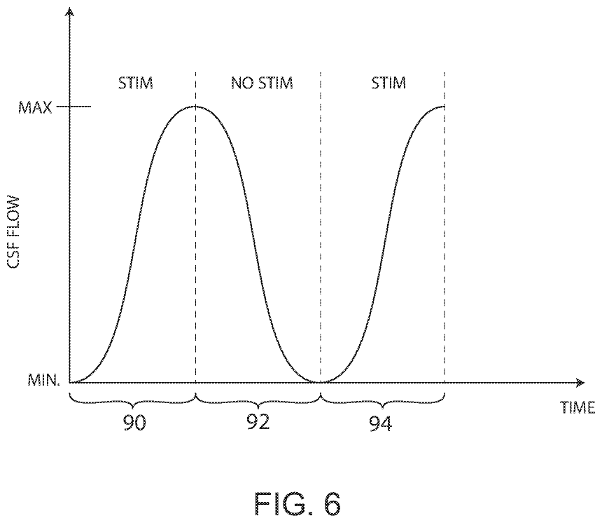

Referring now to FIG. 6, the delivery of the electrical stimulation 72 of the first carrier wave 62a, second carrier wave 62b, and third carrier wave 62c to the electrodes 50 may be used to increase or decrease the flow of CSF through the parenchyma and clearance of CSF from the brain, or to maximize or minimize the flow of CSF through the parenchyma and clearance of CSF from the brain, for different drug delivery profiles.

The increase of CSF clearance from the brain may be desired, such as shown during time periods 90, 94, for example, when it is desired for drugs to either enter or be expelled from the brain at a faster rate. In one embodiment of the present invention, the delivery of the electrical stimulation 72 may be optimized to increase clearance of CSF from the brain by at least 30% and at least 40% and at least 50% above non-stimulation levels.

In contrast, the decrease of CSF clearance from the brain may be desired, such as shown during time period 92, for example, to allow drugs to dwell longer in the brain. In one embodiment of the present invention, the delivery of the electrical stimulation 72 may be stopped or paused to decrease clearance of CSF from the brain by at least 30% and at least 40% and at least 50% below the highest CSF clearance levels. The delivery of the electrical stimulation 72 may increase the half-life of the delivered drug by at least 10% and at least 20% and at least 30% and at least 40% and at least 50% for greater penetration of the drug.

The “dialing up” and “dialing down” of CSF clearance may be used in combination to provide a coordinated effort for certain drug delivery profiles. For example, the CSF clearance may be “dialed up” during the time period 90 to improve penetration of difficult to infiltrate drugs into the brain, but then “dialed down” during the time period 92 to allow for improved uptake of the drug once inside the brain. Once the desired concentration of the drug is obtained within the brain, the CSF clearance may once again be “dialed up” during the time period 94 to quickly clear the drug concentration from the brain.

The following are exemplary embodiments of modulating CSF clearance quickly “up” or “down” for different desired drug delivery profiles and applications.

### Example 6: Increase or “Dial Up” CSF Clearance

The rapid increase of CSF clearance may be desired during the time periods 90, 94 to clear high systemic toxicity drugs or substances from the brain, for example, in the situation of a drug overdose or over delivery. The delivery of the electrical stimulation 72 may be optimized to increase clearance of CSF from the brain by at least 30% and at least 40% and at least 50% higher than non-stimulation levels during the overdose or overexposure of drugs that need to be quickly cleared from the brain. The CSF clearance may also assist with delivery of drugs to the brain which do not readily penetrate the brain.

In one embodiment, the increase of CSF clearance may assist with clearing overexposure to substances such as drugs (e.g., opioids such as morphine, codeine, salvia divinorum, heroin, oxycodone, hydromorphone, hydrocodone, salvanorin A, methadone, buprenorphine, fentanyl and benzodiazepines), hormones (e.g., stress hormones such as adrenaline and cortisol), proteins (e.g., amyloid β (Aβ), apolipoprotein E (APOE), α-synuclein (α-syn), DJ-1, LRRK2, PINK1/PARKIN, tau, C-tau), Huntington protein, superoxide dismutase 1 (SOD1), TAR DNA-binding protein 43 (TDP-43), FUS, progranulin, SCN1A, mutant Huntington protein (mHtt), SOD1 mutant G93A) or other neurotoxic chemicals (e.g., methylmercury, polychlorinated biphenyls, ethanol, lead, arsenic, toluene, manganese, fluoride, chlorpyrifos, tetrachloroethylene, polybrominated diphenyl ethers, and dichlorodiphenyltrichloroethane) and the like from the brain.

In addition, the rapid increase of CSF clearance may be desired to improve the penetration of pharmaceutical drug concentrations into the brain, for example, to improve the delivery of treatments such as medication (e.g., latrepirdine, riluzole, donepezil, galantamine, memantine, rivastigmine, exelon, and/or Levodopa), protein therapy (e.g., protein degradation therapy), and/or immunotherapy (e.g., active vaccination and/or passive vaccination) with poor infiltration into the brain.

### Example 7: Decrease or “Dial Down” CSF Clearance

The decrease of CSF clearance may be desired during the time period 92 in the administration of drugs with non-optimal uptake profiles. The delivery of the electrical stimulation 72 may be optimized to decrease clearance of CSF from the brain by at least 30% and at least 40% and at least 50% below highest CSF clearance levels when it is desired for drugs to linger in the brain for better uptake of the drugs. The delivery of the electrical stimulation 72 may increase the half-life of the delivered drug by at least 10% and at least 20% and at least 30% and at least 40% and at least 50%.

For example, Levodopa (L-dopa) is used to treat the motor symptoms associated with Parkinson's disease, Parkinsonism, dopamine-responsive dystonia, and Parkinson-plus syndrome. These diseases are characterized by a degeneration of dopamine neurons in the substantia nigra that project axons to the striatum, release dopamine and thus influence motor behavior. The resulting dopamine deficits produce the classic motor symptoms such as bradykinesia, akinesia, and tremor. The production of supraphysiologic dopamine concentrations in the brain have been found to lead to cognitive impairments and ultimately impact quality of life of the Parkinson's patient.

L-dopa is the precursor to the neurotransmitters dopamine, norepinephrine (noradrenaline), and epinephrine (adrenaline). L-dopa can cross the BBB via the L-system large neutral amino acid transporter, whereas dopamine cannot. The doping of L-dopa in the brain results in the conversion of L-dopa into dopamine by aromatic amino-acid decarboxylase (AADC) also known as DOPA decarboxylase. L-dopa is typically taken orally, multiple times a day, e.g., three or four times a day, to improve the concentration of dopamine in the brain.

According to the present invention, the delivery of the electrical stimulation 72 may be stopped or paused to decrease clearance of CSF from the brain to non-stimulation levels, e.g., immediately following the administration of L-dopa. By slowing down the clearance of CSF in the brain, doses of L-dopa can linger longer in the brain and increase the uptake profile to the drug.

It is understood that drug delivery profiles may be coupled with electrical stimulation protocols as described herein in order for drugs or substances to linger in the brain longer or cleared faster depending on the desired result. Brain activity may be measured in real-time in order to also determine optimal delivery periods or “therapeutic windows” for drug delivery such as during gamma frequencies when AQP4 channels are more open.

The above described methods may be used to treat patients with for example depression, anxiety and epilepsy by increasing the influx of CSF into the brain parenchyma. It has been found that an increase in CSF into the brain parenchyma further dilutes endogenous concentrations of neurochemical transmitters/bioactive molecules and reduces ephaptic (non-synaptic) coupling implicated in abnormal circuit behaviors associated with multiple disorders of the nervous system, for example, anxiety disorders, epilepsy, Alzheimer's disease, and Parkinson's disease.

It is understood that the present invention is not limited to the treatment of traumatic brain injury/chronic traumatic encephalopathy, epilepsy, Alzheimer's disease, and Parkinson's disease and the like and may also be used to treat other conditions and disorders such as hydrocephalus caused by a buildup of CSF in the brain parenchyma by increasing the clearance of CSF through the brain. Also, clearance of orally administered drugs that cross the blood brain barrier, or drugs/biomolecules that are infused via an injection/catheter, can be modulated by changing the CSF flow rate.

Electrical stimulation of the nerves as described above has been found to induce neuroplasticity or cortical plasticity and introduce and modify brain wave oscillation frequency useful for treating neuro-psychiatric disorders. For example, brain wave oscillations may be increased to natural brain wave frequencies, e.g., 8 to 13 hertz, which may be lower in older adults experiencing memory difficulties, and activation of circuitry through the trigeminal sensory nuclei to create broad neurochemical changes in the brain mediated by cross connectivity to the nucleus of the solitary tract (NTS) to enhance plasticity in many conditions such as stroke and tinnitus. The NTS has inputs to locus coeruleus, raphae nucleus, and nucleus basalis which are responsible for most norepinephrine, serotonin, dopaminergic, and cholinergic projections to the rest of the brain.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “an electronic computer” and “a processor” or “the microprocessor” and “the processor,” can be understood to include one or more of these devices that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

References to “a processor” should be understood to include electronic computers, microprocessors, microcontrollers, FPGA devices, ASIC devices and similar programmable or program defined electronic circuits and collections of such devices that can communicate in a stand-alone and/or a distributed environment(s), and can thus be configured to communicate via wired or wireless communications with other processors. Furthermore, references to memory, unless otherwise specified, can include one or more processor-readable and accessible memory elements and/or components that can be internal to the processor or external to the processor and accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What we claim is:

1. An electrical stimulation device for modulating function of a glymphatic system or meningeal lymphatic system comprising:
an electrical generator generating a first carrier wave having a first carrier frequency and a second carrier wave having a second carrier frequency;
a modulator receiving the second carrier wave and a modulation wave to modulate the second carrier wave to produce a modulated second carrier wave;
an electrical modulation generator generating the modulation wave having a predetermined periodicity providing a first period of stimulation and a second period of no stimulation, the predetermined periodicity selected to increase vessel wall movement over continuous stimulation of the glymphatic or meningeal lymphatic system by the first carrier wave; and
a nerve stimulator configured to stimulate a cranial nerve and receiving the first carrier wave and the modulated second carrier wave simultaneously to stimulate the glymphatic or meningeal lymphatic system system to modulate cerebral spinal fluid (CSF)/interstitial fluid (ISF) flow.

2. The electrical stimulation device of claim 1 wherein the first carrier wave has a frequency promoting increased function of the aquaporin-4 water channel system.

3. The electrical stimulation device of claim 1 wherein the frequency of the first carrier wave is at typical delta band brain wave (0.1-4 Hz) or lower frequencies.

4. The electrical stimulation device of claim 1 wherein the frequency of the second carrier wave is greater than the frequency of the first carrier wave.

5. The electrical stimulation device of claim 1 wherein a duty cycle of the modulation wave is between 10% and 50%.

6. The electrical stimulation device of claim 1 wherein the modulation wave has a pulse duration that is at least twice as long as a pulse interval.

7. The electrical stimulation device of claim 1 wherein the modulated second carrier wave has a duty cycle modulating vessel wall movement in the brain.

8. The electrical stimulation device of claim 1 wherein the electrical generator generates a third carrier wave and the at least one electrode receives the third carrier wave before or between periods of receiving the first carrier wave and modulated second carrier wave.

9. The electrical stimulation device of claim 8 wherein the third carrier wave has a third carrier frequency promoting phagocytic phenotype in glial cells to break down waste biomolecules and misfolded proteins.

10. The electrical stimulation device of claim 8 wherein the third carrier wave is at a frequency that is greater than a frequency of the first carrier wave.

11. The electrical stimulation device of claim 8 wherein the frequency of the third carrier wave is at gamma brain wave frequency.

12. The electrical stimulation device of claim 1 wherein at least one electrode is adapted to stimulate at least one of a trigeminal nerve, one of a facial nerve, buccal branch nerve, mental branch nerve and facial branch nerve.

13. A method of modifying function of a glymphatic system or meningeal lymphatic system comprising:
positioning at least one electrode in close proximity to a nerve;
generating a first carrier wave having a first carrier frequency stimulating the glymphatic system or meningeal lymphatic system into increased cerebral spinal fluid (CSF)/interstitial fluid (ISF) flow;
generating a second carrier wave having a second carrier frequency stimulating the glymphatic or meningeal lymphatic system into increased cerebral spinal fluid (CSF)/interstitial fluid (ISF) flow;
generating a modulation wave having a predetermined periodicity providing a first period of stimulation and a second period of modified or no stimulation, the predetermined periodicity selected to increase vessel wall movement over continuous or no stimulation of the glymphatic or meningeal lymphatic system by the first carrier wave;
modulating the second carrier wave by applying the modulation wave to the second carrier wave; and
applying the first and second carrier wave to the at least one electrode simultaneously.

14. The method of claim 13 wherein the first carrier wave frequency of the first carrier wave is at typical delta band brain wave (0.1-4 Hz) or lower frequencies

15. The method of claim 13 wherein the second carrier frequency of the second carrier wave is greater than the first carrier frequency of the first carrier wave.

16. The method of claim 13 wherein a duty cycle of the modulation wave is between 10% and 50%.

17. The method of claim 13 wherein the modulation wave has a pulse duration that is at least twice as long as a pulse interval.

18. The method of claim 16 further comprising generating a third carrier wave before or between periods of generating the first carrier wave and second carrier wave simultaneously and applying the third carrier wave to the electrode.

19. The method of claim 18 wherein the third carrier wave has a third carrier frequency promoting phagocytic phenotype in glial cells to break down waste biomolecules and misfolded proteins.

20. The method of claim 18 wherein the first carrier wave and second carrier wave are applied to a first electrode and the third carrier wave is applied to a second electrode wherein the first electrode and second electrode are positioned at different locations to stimulate different nerves.

21. A method of modifying fluid and molecule movement through a glymphatic or meningeal lymphatic system system comprising:
positioning at least one electrode in close proximity to a nerve;
generating a carrier wave having a carrier frequency stimulating the glymphatic or meningeal lymphatic system into a modified cerebral spinal fluid (CSF)/interstitial fluid (ISF) flow speed;
generating a modulation wave having a predetermined periodicity providing a first period of stimulation of the glymphatic or meningeal lymphatic system and a second period of relaxation of the glymphatic or meningeal lymphatic system, the predetermined periodicity selected to modify fluid and molecular movement over continuous or no stimulation of the glymphatic or meningeal lymphatic system by the carrier frequency;
modulating the carrier wave by applying the modulation wave to the at least one of the carrier wave; and
selectively applying the carrier wave to the at least one electrode wherein the carrier wave is delivered to the electrode when it is desired to increase fluid and molecular movement and the carrier wave is not delivered to the electrode when it is desired to decrease fluid and molecular movement.
